(19) United States
(12) Patent Application Publication
(10) Pub. No.: US 2011/0071428 A1
(43) Pub. Date: Mar. 24, 2011

(54) BIOPSY DEVICE

(75) Inventors: Mary Frecker, State College, PA (US); Abraham Mathew, Hershey, PA (US); Casandra Niebel, State College, PA (US); Andrew Rau, Jackson Center, PA (US)

(73) Assignee: The Penn State Research Foundation, University Park, PA (US)

(21) Appl. No.: 12/871,344

(22) Filed: Aug. 30, 2010

Related U.S. Application Data
(63) Continuation-in-part of application No. 12/534,329, filed on Aug. 3, 2009.
(60) Provisional application No. 61/085,506, filed on Aug. 1, 2008.
(60) Provisional application No. 61/150,568, filed on Feb. 6, 2009.
(60) Provisional application No. 61/237,959, filed on Aug. 28, 2009.

(51) Int. Cl. A61B 10/00

(52) U.S. Cl. 600/566

(57) ABSTRACT

Biopsy devices which can be used with a flexible endoscope are described herein. Inventive biopsy devices can be used in minimally invasive procedures and other biopsy applications. Inventive biopsy devices can be used through an endoscope channel to perform zero invasive biopsies on the gastrointestinal system and other organs. Actuation devices for use to twist or manipulate the biopsy device even when it is at the end of the endoscopic cable are provided according to embodiments of the present invention.

### REFERENCE TO RELATED APPLICATIONS

This application is a continuation-in-part of U.S. patent application Ser. No. 12/534,329, filed Aug. 3, 2009, which claims priority to U.S. Provisional Patent Application Ser. Nos. 61/085,506, filed Aug. 1, 2008, and 61/150,568, filed Feb. 6, 2009, This application claims priority to U.S. Provisional Patent Application Ser. No. 61/237,959, filed Aug. 28, 2009. The entire content of each application is incorporated herein by reference.

### FIELD OF THE INVENTION

The present invention relates generally to biopsy devices. In specific embodiments, biopsy devices for use in endoscopic procedures are described.

### BACKGROUND OF THE INVENTION

A variety of fields would benefit from the size reduction of mechanical structures.

In a particular example, improved biopsy devices are required. When using a biopsy device, a sufficient number of cells to perform an adequate examination must be removed. If enough cells are not removed, another biopsy must be performed, increasing tissue damage, patient discomfort, recovery time, and risk of infection.

The requirement for adequate biopsy material must be balanced against the requirements of patient care. Demand for surgical procedures which drastically reduce patient recovery time and discomfort has led to a paradigm shift in modern medicine. Minimally invasive surgery (MIS) is a rapidly developing medical practice and is constantly being advanced through the introduction of novel experimental procedures. MIS procedures require smaller external incisions than traditional surgery, or are completely devoid of external incisions. The benefits of such procedures from a patient care perspective include less discomfort and scaring, shortened recovery time and decreased chance of surgical infections. These benefits have caused an extremely high demand for MIS over traditional surgery, resulting in the introduction of several groundbreaking procedures.

A procedure known as natural orifice transluminal endoscopic surgery (NOTES) is particularly promising as a minimally invasive technique. During NOTES, the surgeon passes a flexible endoscope through a natural orifice, e.g. the mouth or anus, in order to access an internal surgical site.

A novel NOTES procedure for transgastric access is currently under development. During this experimental procedure, the endoscope is inserted through the esophagus and a small internal incision is made in the inner lining of the stomach. The surgeon then passes a forceps tool through the endoscope and tunnels between the layers of the stomach wall, subsequently making a second incision in the outer layer of the stomach. Tunneling between the layers of the stomach wall creates an effective seal between the acidic inside of the stomach and the abdominal cavity, preventing unwanted leakage. After the procedure has concluded, no external incisions are present, and the internal incisions are sutured.

These and other surgical techniques are used to obtain biopsy material. There is a continuing need for biopsy devices that provide adequate amounts of biopsy material, minimal tissue damage, and which do not buckle or break due to contact with tissue.

### SUMMARY OF THE INVENTION

Biopsy devices according to embodiments of the present invention include an elongated member having an external surface, a longitudinal axis, a distal end and a proximal end, and at least two protuberances extending from the elongated member, the protuberances oriented to provide low resistance entry of the distal end into a tissue or biological material and to provide higher resistance on withdrawal of the distal end from the tissue or biological material.

Biopsy devices according to embodiments of the present invention include an elongated member having a non-hollow cross-section along the length of the longitudinal axis, an external surface, a longitudinal axis, a distal end and a proximal end, and at least two protuberances extending from the elongated member, the protuberances oriented to provide low resistance entry of the distal end into a tissue or biological material and to provide higher resistance on withdrawal of the distal end from the tissue or biological material.

Biopsy devices according to embodiments of the present invention include an elongated member having a longitudinal axis, an external surface, a longitudinal axis, a distal end and a proximal end, and at least two protuberances extending from the elongated member, wherein the elongated member includes a working portion disposed towards the distal end of the elongated member and a support portion disposed proximally from the working portion, wherein the protuberances are disposed on the working portion of the elongated member, the protuberances oriented to provide low resistance entry of the distal end into a tissue or biological material and to provide higher resistance on withdrawal of the distal end from the tissue or biological material.

Biopsy devices according to embodiments of the present invention include an elongated member having a longitudinal axis, an external surface, a longitudinal axis, a distal end and a proximal end, and at least two protuberances extending from the elongated member, wherein the elongated member includes a working portion disposed towards the distal end of the elongated member and a support portion disposed proximally from the working portion, wherein the working portion of the elongated member is non-hollow, wherein the protuberances are disposed on the working portion of the elongated member, the protuberances oriented to provide low resistance entry of the distal end into a tissue or biological material and to provide higher resistance on withdrawal of the distal end from the tissue or biological material.

Biopsy devices according to embodiments of the present invention include an elongated member having an external surface, a longitudinal axis, a distal end and a proximal end, and at least two protuberances extending from the elongated member, the protuberances oriented to provide low resistance entry of the distal end into a tissue or biological material and to provide higher resistance on withdrawal of the distal end from the tissue or biological material, wherein the elongated member is attached to a support, such as a flexible shaft, inserted in an accessory channel of an endoscope.

Biopsy devices according to embodiments of the present invention include an elongated member having an external surface, a longitudinal axis, a distal end and a proximal end, and at least two protuberances extending from the elongated member, wherein the protuberances each have a front edge which tapers towards the distal end of the elongated member and a rear edge, a forward segment of the elongated member defined between the rear edge of each protuberance and the distal end and a rearward segment of the elongated member defined between the rear edge of each protuberance and the proximal end, wherein the rear edge is disposed so that at least a portion of the rear edge forms an angle which is 90° or less with the rearward segment, providing resistance when the biopsy device is removed from a bodily substance.

Biopsy devices according to embodiments of the present invention include an elongated member having an external surface, a longitudinal axis, a distal end and a proximal end, and at least two protuberances extending from the elongated member, wherein the protuberances each have a top edge and a bottom edge, wherein the protuberances each have a longitudinal axis that is substantially parallel to the longitudinal axis of the elongated member, wherein the top and bottom edges of the protuberances extend from the elongated member, defining a space between the bottom edge of each protuberance and the external surface of the elongated member, providing resistance when the biopsy device is rotated in a bodily substance, thereby collecting biopsy material in the space and/or on the bottom edge.

Biopsy devices according to embodiments of the present invention include an elongated member having a longitudinal axis, an external surface, a longitudinal axis, a distal end and a proximal end, and at least two protuberances extending from the elongated member, wherein the elongated member includes a working portion disposed towards the distal end of the elongated member and a support portion disposed proximally from the working portion, wherein the working portion has a length in the range of about 1-14 millimeters, inclusive and a total diameter of the elongated member and protuberances in the range of about 0.5-6 millimeters, inclusive and wherein the protuberances are disposed on the working portion of the elongated member, the protuberances oriented to provide low resistance entry of the distal end into a tissue or biological material and to provide higher resistance on withdrawal of the distal end from the tissue or biological material.

Biopsy devices are provided according to embodiments of the present invention which include an elongated member having an external surface, a longitudinal axis, a distal end and a proximal end, and at least one helical protuberance extending radially from the elongated member, wherein the helical protuberance has a front edge which tapers towards the distal end of the elongated member and a rear edge, wherein the rear edge of the helical protuberance is disposed to form an angle which is 90° or less with the longitudinal axis of the elongated member, providing resistance when the biopsy device is removed from a bodily substance.

Methods of obtaining a sample of biological material are provided according to embodiments of the present invention which include inserting into a biological material to be sampled a biopsy device having an elongated member having an external surface, a longitudinal axis, a distal end and a proximal end, and at least two protuberances extending from the elongated member, the protuberances oriented to provide low resistance entry of the distal end into the biological material and to provide higher resistance on withdrawal of the distal end from the biological material; and withdrawing the biopsy device.

Methods of obtaining a sample of biological material are provided according to embodiments of the present invention which include inserting into a biological material to be sampled a biopsy device having an elongated member having an external surface, a longitudinal axis, a distal end and a proximal end, and at least two protuberances extending from the elongated member, the protuberances oriented to provide low resistance entry of the distal end into the biological material and to provide higher resistance on rotation of the distal end in the biological material; rotating the biopsy device in the biological material and withdrawing the biopsy device. According to embodiments of the present invention, the biopsy device is not rotated during insertion of the biopsy device into the biological material.

Methods for forming a polycrystalline sintered mesoscale biopsy device are provided according to the present invention which include filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hmin:Tmin of 20-80:1:0.5-10; then removing said photoresist; and heating said mold fill to remove said polymer.

Methods for forming a polycrystalline sintered mesoscale biopsy device are provided according to the present invention which include filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hmin:Tmin of 20-80:1:0.5-10; then removing said photoresist; and heating said mold fill to remove said polymer, wherein said slurry of particles have a particle diameter of from 5 nanometers to 500 microns and said mold cavity has a ratio of segment dimensions L:Hmin:Tmin of 20-80:1:0.5-10.

Methods for forming a polycrystalline sintered mesoscale biopsy device are provided according to the present invention which include filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hmin:Tmin of 20-80:1:0.5-10; then removing said photoresist; and heating said mold fill to remove said polymer, wherein said slurry of particles have a particle diameter of from 10 nanometers to 500 microns.

Methods for forming a polycrystalline sintered mesoscale biopsy device are provided according to the present invention which include filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hmin:Tmin of 20-80:1:0.5-10; then removing said photoresist; and heating said mold fill to remove said polymer, wherein said monomer is present from 0.5 to 20 weight percent of the weight of said particles.

Methods for forming a polycrystalline sintered mesoscale biopsy device are provided according to the present invention which include filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hmin:Tmin of 20-80:1:0.5-10; then removing said photoresist; and heating said mold fill to remove said polymer, wherein said removing said photoresist is by reactive ion etching.

Methods for forming a polycrystalline sintered mesoscale biopsy device further include removal of mold overburden prior to said removing said photoresist according to embodiments of the present invention.

Methods for forming a polycrystalline sintered mesoscale biopsy device further include filling a second mold cavity formed in a second photoresist with a second slurry of particles of ceramic, metal, or a combination thereof and a second polymerizable monomer; removing said second photoresist; contacting said mold fill with said second mold fill to form an interface; and heating said second mold fill in contact with said mold fill to sinter said mold fill with said second mold fill according to embodiments of the present invention.

Methods for forming a polycrystalline sintered mesoscale biopsy device further include filling a third mold cavity formed in a third photoresist with a third slurry of particles of ceramic, metal, or a combination thereof and a third polymerizable monomer; removing said third photoresist; contacting said mold fill with said third mold fill to form a second interface; and heating said third mold fill in contact with said second mold fill to sinter said second mold fill with said third mold fill according to embodiments of the present invention.

### BRIEF DESCRIPTION OF THE INVENTION

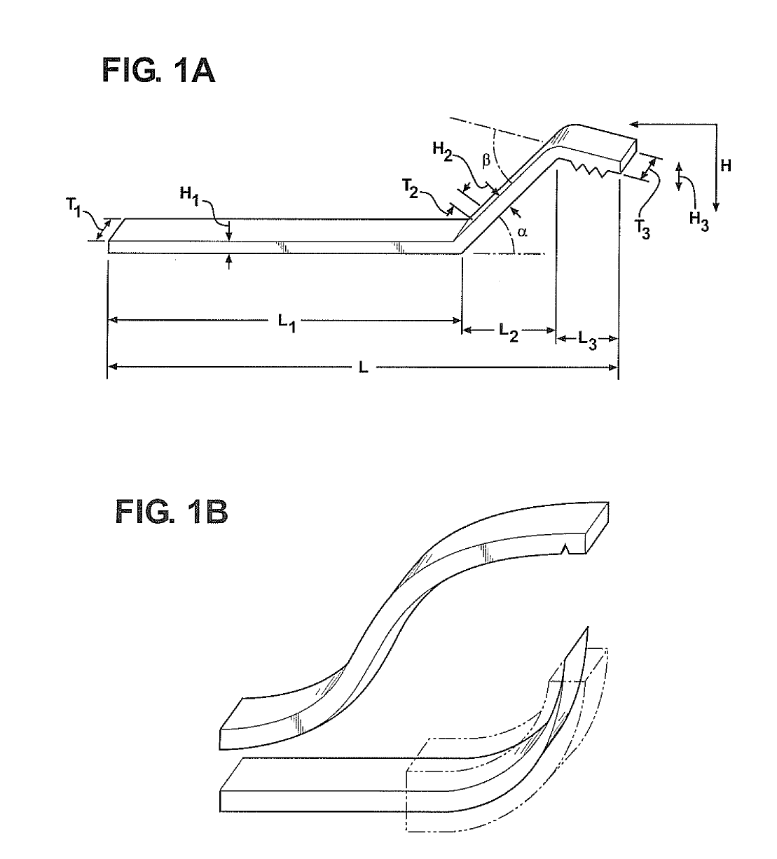

FIG. 1A is a perspective view of an inventive component;

FIG. 1B is a perspective view of a pair representative inventive components forming an anvil and cutter pair, the cutter having a lap joint with a reinforcing portion;

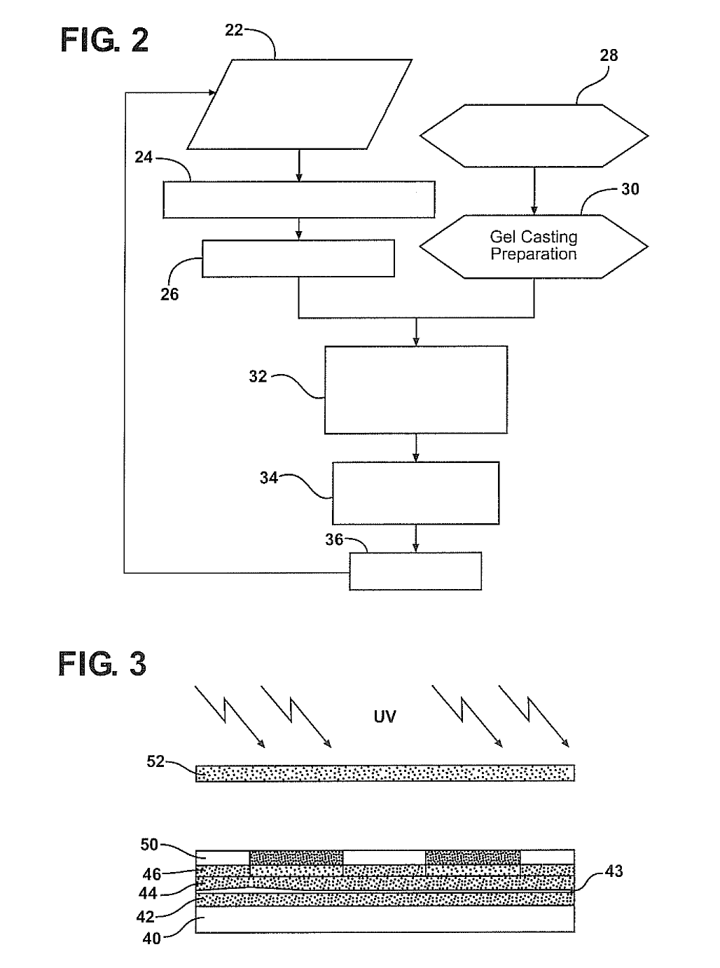

FIG. 2 is a schematic flowchart of a process for design and manufacture of an inventive component;

FIG. 3 is a cross-sectional view of a fabrication mold for the inventive component;

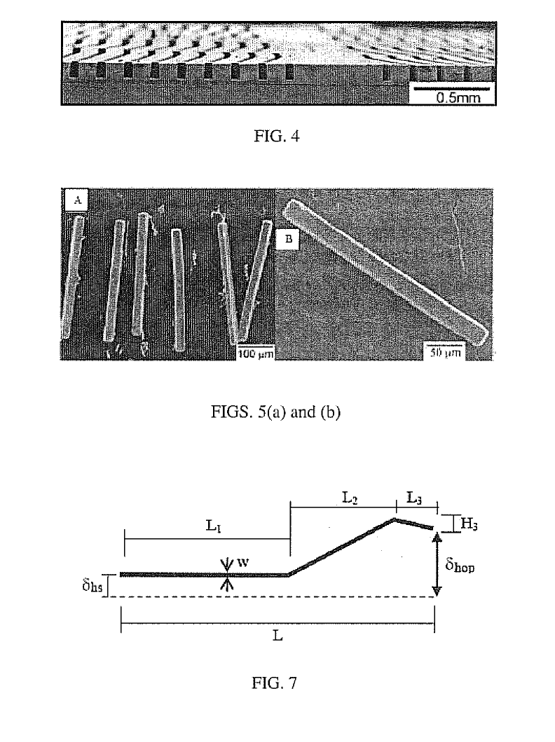

FIG. 4 is a cross-sectional scanning electron micrograph (SEM) of the fabrication mold;

FIGS. 5A and 5B are SEMs of zirconia test bars (A) and a cleaned, magnified test bar (B) as shown in (A);

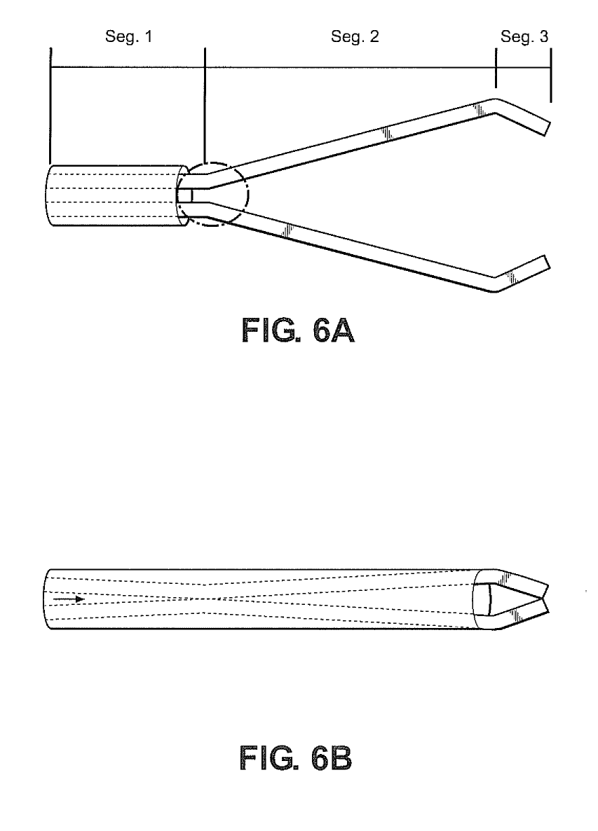

FIGS. 6A and 6B are a schematic of a component of FIG. 1A as part of a pair of micro forceps in open (A) and closed (B) configurations;

FIG. 7 is a plan view of an inventive component of FIG. 1A as a micro forceps part annotated to show additional geometric variables;

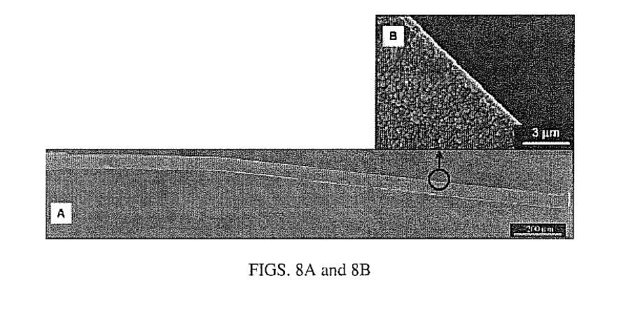

FIGS. 8A and 8B are an SEM of an inventive component of FIG. 7 and a magnified SEM of edge resolution from FIG. 8A (B), teeth on the segment 3 are noted;

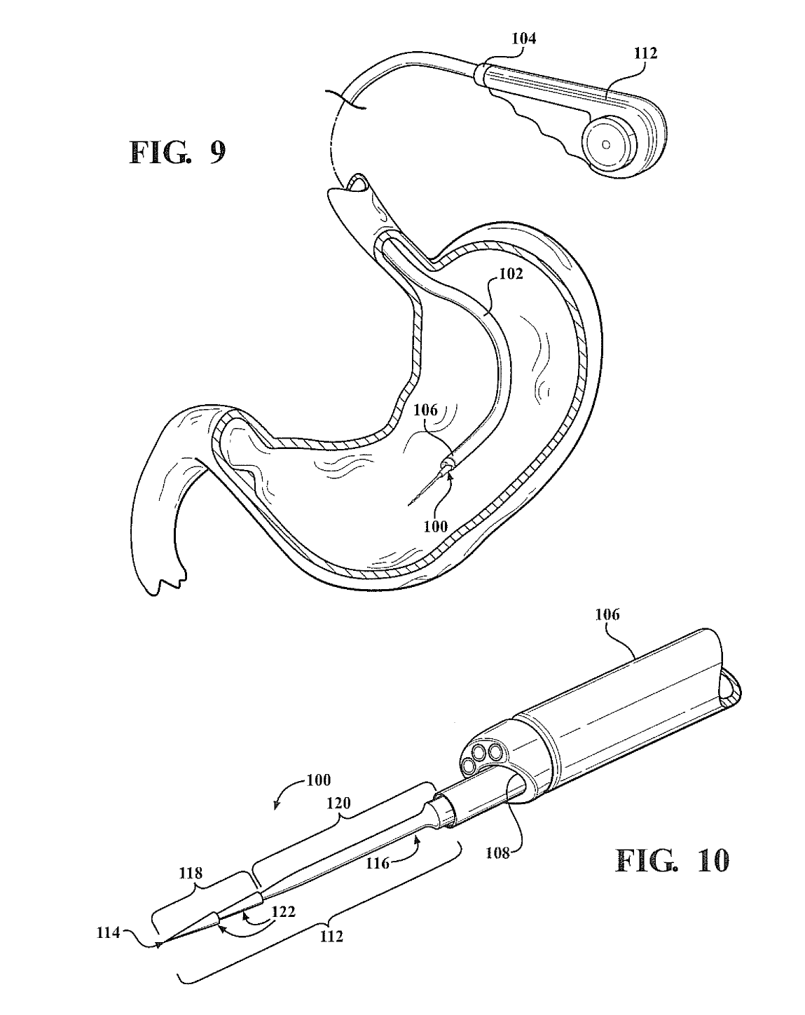

FIG. 9 is a perspective view of a biopsy device according to an embodiment of the present invention disposed through an endoscope, with the endoscope extending into the stomach of a surgical patient;

FIG. 10 is a perspective view of a biopsy device according to an embodiment of the present invention extending from the end of an endoscope;

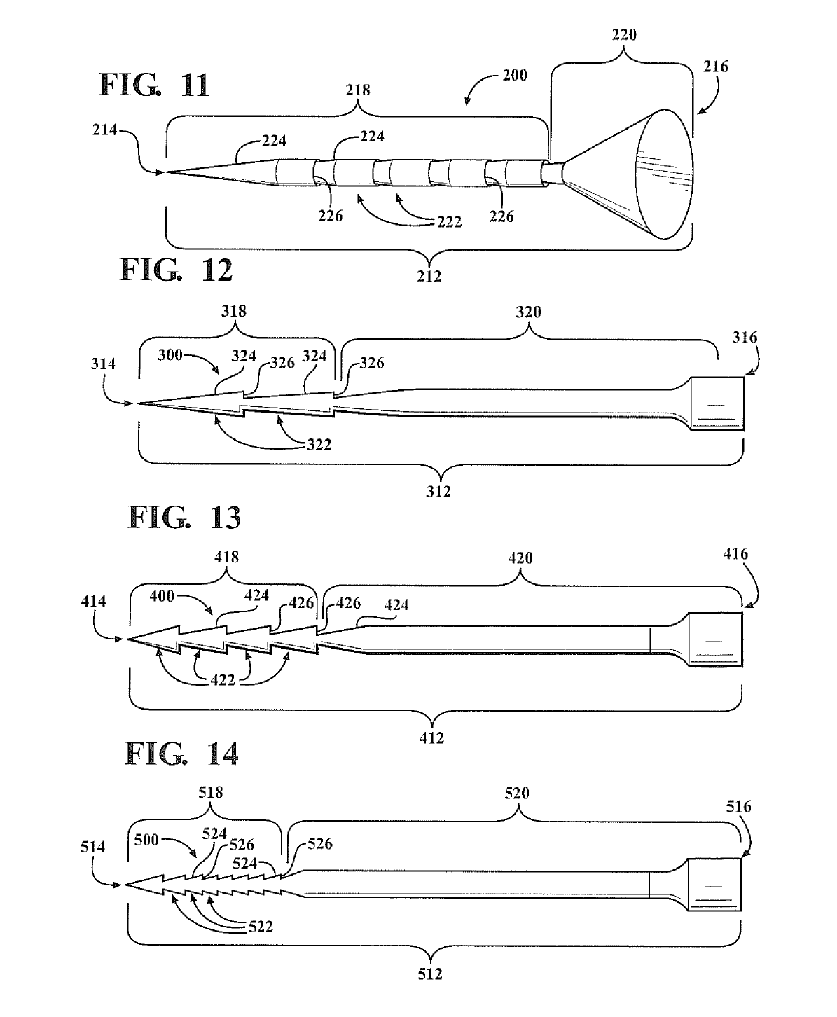

FIG. 11 is a perspective view of a biopsy device according to an embodiment of the present invention;

FIG. 12 is a perspective view of a biopsy device according to an embodiment of the present invention;

FIG. 13 is a perspective view of a biopsy device according to an embodiment of the present invention;

FIG. 14 is a perspective view of a biopsy device according to an embodiment of the present invention;

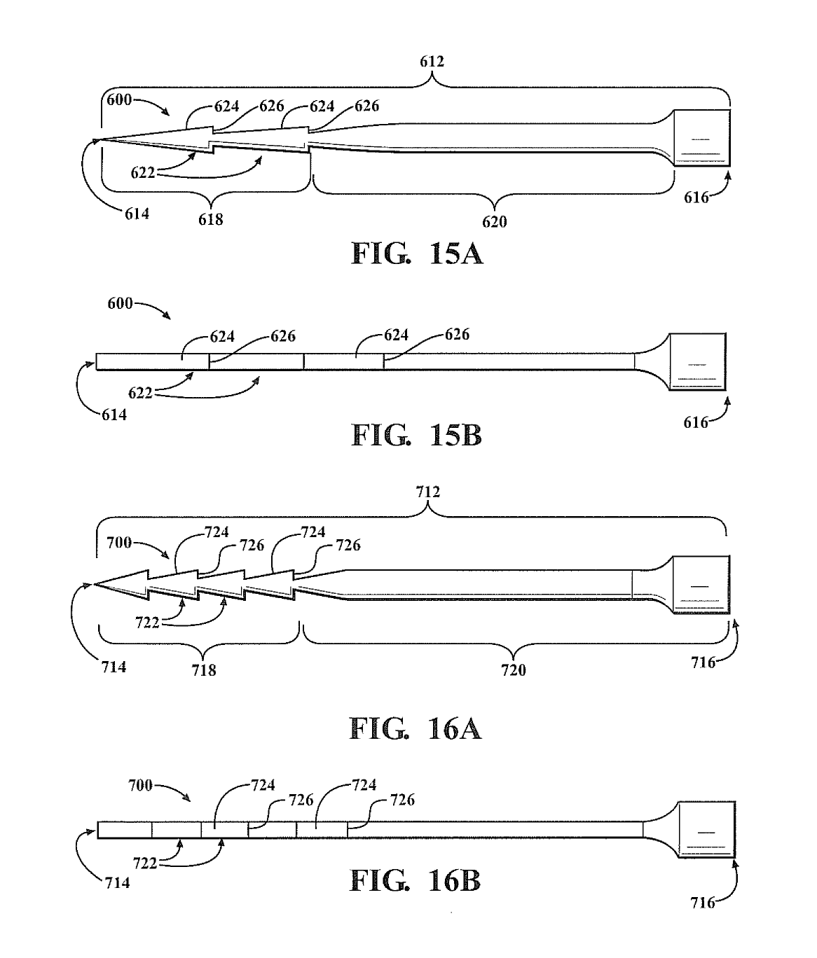

FIG. 15A is a frontal view of a biopsy device according to an embodiment of the present invention;

FIG. 15B is a side view of the biopsy device of FIG. 3E according to an embodiment of the present invention;

FIG. 16A is a frontal view of a biopsy device according to an embodiment of the present invention;

FIG. 16B is a side view of the biopsy device of FIG. 3G according to an embodiment of the present invention;

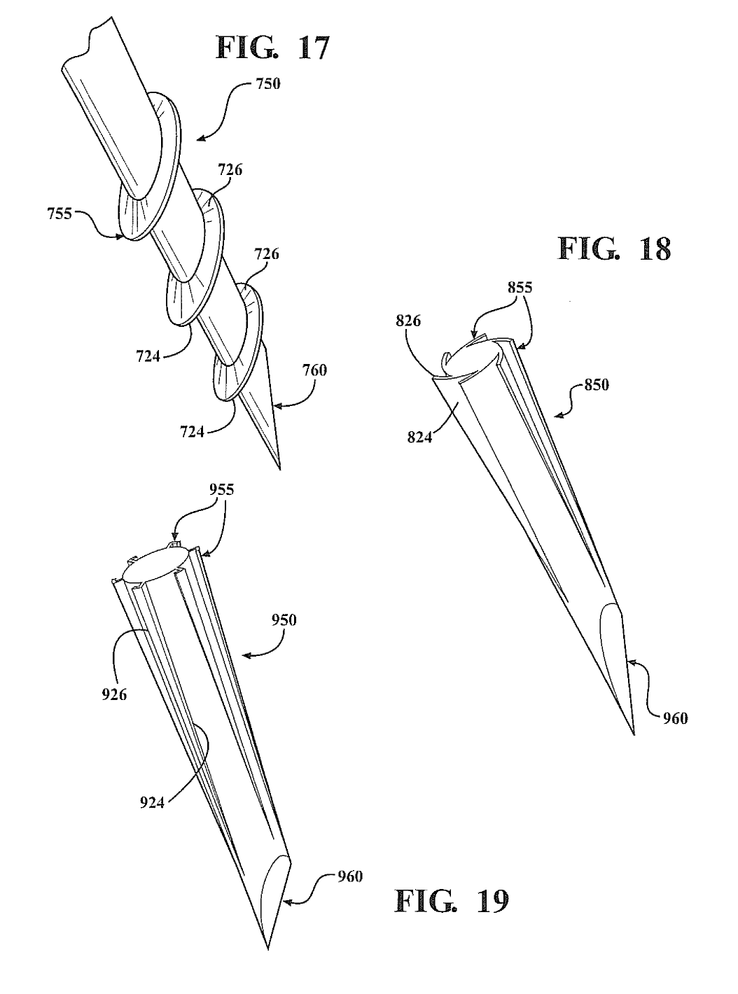

FIG. 17 is a perspective view of the working end of a biopsy device according to an embodiment of the present invention;

FIG. 18 is a perspective view of the working end of a biopsy device according to an embodiment of the present invention;

FIG. 19 is a perspective view of the working end of a biopsy device according to an embodiment of the present invention;

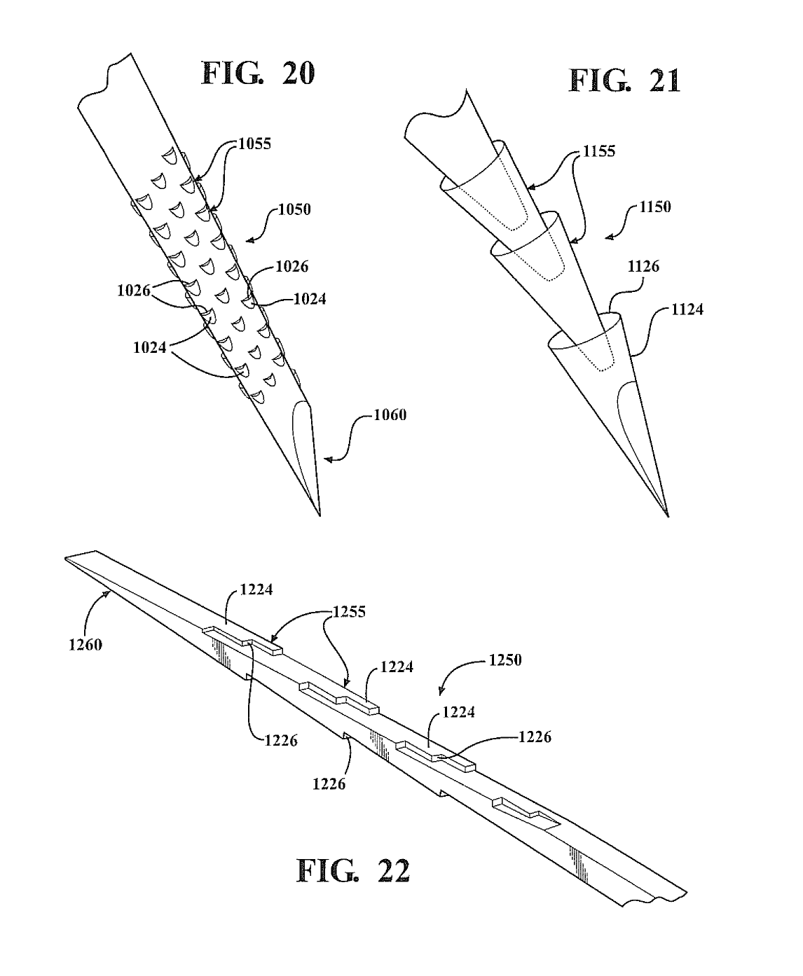

FIG. 20 is a perspective view of the working end of a biopsy device according to an embodiment of the present invention;

FIG. 21 is a perspective view of the working end of a biopsy device according to an embodiment of the present invention; and

FIG. 22 is a perspective view of a multilayer biopsy device according to an embodiment of the present invention.

### DETAILED DESCRIPTION OF THE INVENTION

Biopsy devices of the present invention provide adequate amounts of biopsy material, minimal tissue damage, and do not buckle or break due to contact with tissue. It is an aspect of biopsy devices described herein that the size parameters of the biopsy device contribute to the ability of the biopsy device to provide adequate amounts of biopsy material, minimal tissue damage, and withstand: 1) insertion into a tissue, 2) manipulation in the tissue if necessary to collect cells and 3) withdrawal from a tissue, without buckling or breaking.

Biopsy devices according to embodiments of the present invention are characterized by easy insertion into a bodily substance, such as a tumor, tissue or organ, and resistance during removal from the bodily substance, or rotation in the bodily substance, in order to capture biopsy material, such as cells. Biopsy devices according to embodiments of the present invention maximize the amount of material removed from the patient, minimize the force to remove the material, and minimize cost.

Biopsy devices according to embodiments of the present invention include an elongated member having a longitudinal axis, a distal end and a proximal end, and at least two protuberances extending from the elongated member. The protuberances each have a front edge which tapers towards the distal end of the elongated member providing low resistance and easy insertion into a bodily substance. The protuberances each have a rear edge. The rear edge of each protuberance defines a segment of the longitudinal axis of the elongated member forward of the rear edge towards the distal end of the elongated member and a segment of the longitudinal axis of the elongated member behind the rear edge towards the proximal end of the elongated member. The rear edge is disposed so that at least a portion of the rear edge forms an angle which is 90° or less with the segment of the longitudinal axis of the elongated member present behind the rear edge towards the proximal end of the elongated member, providing resistance when the biopsy device is removed from a bodily substance.

In further embodiments, the protuberances each have a top edge and a bottom edge each of which tapers towards the distal end of the elongated member providing low resistance and easy insertion into a bodily substance. The top and bottom edges of each protuberance each have a longitudinal axis that is substantially parallel to the longitudinal axis of the elongated member. The top and bottom edges of the protuberances extend from the elongated member, defining a space between the bottom edge of each protuberance and the surface of the elongated member, providing resistance when the biopsy device is rotated in a bodily substance, thereby collecting biopsy material in the space and/or on the bottom edge.

In further embodiments, biopsy devices according to embodiments of the present invention include an elongated member having a longitudinal axis, a distal end and a proximal end, and

The elongated member is preferably non-hollow along the length of the longitudinal axis according to embodiments of the present invention.

In further embodiments, the elongated member includes a working portion and a support portion. The working portion is disposed towards the distal end of the elongated member and has at least two protuberances extending from the elongated member. The support portion is disposed towards the proximal end of the elongated member and is devoid of protuberances extending from the elongated member. In preferred embodiments, the working portion of the elongated member and the support portion of the elongated member is non-hollow. Optionally, the working portion of the elongated member is non-hollow and the support portion of the elongated member is hollow.

Biopsy devices according to preferred embodiments of the present invention include a working portion having a length in the range of about 1-14 millimeters, inclusive. At least two protuberances extend from the working portion and the total diameter of the elongated member and protuberances is in the range of about 0.5-6 millimeters, inclusive. The support portion can have any convenient length or width for the intended application or subject.

In use, at least a portion of the elongated member having protuberances extending therefrom is inserted into a tissue of a living subject to be sampled and manipulated such that a portion of the tissue is deposited on at least one of the protuberances.

According to embodiments of the present invention, the protuberances are oriented to provide low resistance entry of the distal end of the elongated member into a tissue or bodily substance and to provide higher resistance on withdrawal of the distal end from the tissue or bodily substance such that a portion of the tissue is deposited on at least one of the protuberances during withdrawal of the elongated member.

According to embodiments of the present invention, the protuberances are oriented to provide low resistance entry of the distal end of the elongated member into a tissue or bodily substance and to provide higher resistance upon rotation of the distal end in the tissue or bodily substance such that a portion of the tissue is deposited on at least one of the protuberances during rotation of the elongated member.

As indicated above, according to embodiments of the present invention, biopsy devices include at least two protuberances extending from the elongated member. The protuberances can be regularly or irregularly spaced along the longitudinal axis of the elongated member.

According to embodiments of the present invention, each protuberance tapers towards the distal end of the elongated member providing lower resistance upon insertion of the biopsy device into a tissue or other solid or semi-solid substance and greater resistance when retracting the biopsy device from the tissue or other solid or semi-solid substance.

In particular embodiments, the protuberances are tooth-like such that the elongated member is serrated.

Biopsy devices according to embodiments of the present invention can have various shapes, sizes and dimensions. For example, inventive biopsy devices can have two- or three-dimensional shapes. In further embodiments, biopsy devices are provided having shapes which have depth, but only have features in two dimensions. This is sometimes referred to as 2.5 D.

The present invention provides biopsy devices that can be used with an endoscope to perform biopsies in zero invasive procedures.

Numerous endoscopes are known. Endoscopes are generally characterized by an elongated flexible insertion portion which can be inserted into a patient's body. The elongated flexible insertion portion generally includes a functional distal end, for instance having an attached imaging device and/or surgical tool, such as a biopsy device. A controller disposed at the proximal end of the elongated flexible insertion portion and one or more actuators is included for control of the functional distal end by a user.

FIG. 9 shows an endoscope 102 including a biopsy device 100 of the present invention. The endoscope extends between two ends 104 and 106 with the end 106 inserted into the body of a subject. The illustrated endoscope includes a handle or control, such as shown at 112. A biopsy device is inserted into an accessory channel of the control head of the endoscope according to embodiments of the present invention.

A detailed view of the end 106 is provided in FIG. 10. As shown, the endoscope has a working channel 108 defined therethrough. Typically, this channel has a diameter in the range of 0.5 to 6 millimeters, and the diameter can be smaller or larger depending on the application.

FIG. 10 shows an embodiment of a biopsy device of the present invention, 100. The illustrated biopsy device includes an elongated member 112 having a distal end 114, a proximal end 116 and having a working portion 118 and a support portion 120. Working portion 118 has two protuberances 122, each of which tapers towards the distal end of the elongated member 114.

FIG. 11 shows an embodiment of a biopsy device of the present invention, 200. The illustrated biopsy device includes an elongated member 212 having a distal end 214, a proximal end 216 and having a working portion 218 and a support portion 220. Working portion 218 has multiple protuberances 222, each of which tapers towards the distal end of the elongated member 214. Each of the protuberances 222 has a front edge 224 and a rear edge 226.

FIG. 12 shows an embodiment of a biopsy device of the present invention, 300. The illustrated biopsy device includes an elongated member 312 having a distal end 314, a proximal end 316 and having a working portion 318 and a support portion 320. Working portion 318 has multiple protuberances 322, each of which tapers towards the distal end of the elongated member 314. Each of the protuberances 322 has a front edge 324 and a rear edge 326.

FIG. 13 shows an embodiment of a biopsy device of the present invention, 400. The illustrated biopsy device includes an elongated member 412 having a distal end 414, a proximal end 416 and having a working portion 418 and a support portion 420. Working portion 418 has multiple protuberances 422, each of which tapers towards the distal end of the elongated member 414. Each of the protuberances 422 has a front edge 424 and a rear edge 426.

FIG. 14 shows an embodiment of a biopsy device of the present invention, 500. The illustrated biopsy device includes an elongated member 512 having a distal end 514, a proximal end 516 and having a working portion 518 and a support portion 520. Working portion 518 has multiple protuberances 522, each of which tapers towards the distal end of the elongated member 514. Each of the protuberances 522 has a front edge 524 and a rear edge 526.

FIG. 15A shows an embodiment of a biopsy device of the present invention, 600. The illustrated biopsy device includes an elongated member 612 having a distal end 614, a proximal end 616 and having a working portion 618 and a support portion 620. Working portion 618 has multiple protuberances 622, each of which tapers towards the distal end of the elongated member 614. Each of the protuberances 622 has a front edge 624 and a rear edge 626. Biopsy device 600 has features in two dimensions and is relatively featureless in the third dimension as shown in the side view of this device, FIG. 15B.

FIG. 16A shows an embodiment of a biopsy device of the present invention, 700. The illustrated biopsy device includes an elongated member 712 having a distal end 714, a proximal end 716 and having a working portion 718 and a support portion 720. Working portion 718 has multiple protuberances 722, each of which tapers towards the distal end of the elongated member 714. Each of the protuberances 722 has a front edge 724 and a rear edge 726. Biopsy device 700 has features in two dimensions and is relatively featureless in the third dimension as shown in the side view of this device, FIG. 16B.

FIG. 17 shows a working portion 750 of an embodiment of a biopsy device of the present invention having a radially disposed protuberance 755 which tapers towards distal end 760. The protuberance 755 has a front edge 724 and a rear edge 726.

FIG. 18 shows a working portion 850 of an embodiment of a biopsy device of the present invention having multiple protuberances 855 which taper towards distal end 860. Each of the protuberances 855 has a top edge 824 and a bottom edge 826.

FIG. 19 shows a working portion 950 of an embodiment of a biopsy device of the present invention having multiple protuberances 955 which taper towards distal end 960. Each of the protuberances 955 has a top edge 924 and a bottom edge 926.

FIG. 20 shows a working portion 1050 of an embodiment of a biopsy device of the present invention having multiple protuberances 1055 which tapers towards distal end 1060. Each of the protuberances 1055 has a front edge 1024 and a rear edge 1026.

FIG. 21 shows a working portion 1150 of an embodiment of a biopsy device of the present invention having multiple protuberances 1155 which tapers towards distal end 1160. Each of the protuberances 1155 has a front edge 1124 and a rear edge 1126.

FIG. 22 shows a working portion 1250 of an embodiment of a biopsy device of the present invention having multiple protuberances 1255 which tapers towards distal end 1260. Each of the protuberances 1255 has a front edge 1224 and a rear edge 1226.

Biopsy devices of the present invention are made of any of various materials used in manufacture of surgical tools, including both rigid and flexible materials, exemplified by, but not limited to, surgical steel and flexible plastics.

Any of various well-known methods can be used to manufacture biopsy devices of the present invention. Wire electrical discharge machining (EDM) can be used for generated of “2.5 D” embodiments of inventive biopsy devices.

A UV lithography process with layered molds is used in order to create three dimensional biopsy needles according to particular embodiments. For example, a rapid forming infiltration process can be used where molds are fabricated using UV lithography, filled with a particulate slurry, and sintered to leave free standing parts. Metal powders or particulate ceramics can be used in this process. Such processes, described in detail hereinbelow, allow creation a biopsy needle of the present invention having multiple layers, such as shown in 22.

Particular methods used to manufacture a biopsy device according to embodiments of the present invention are described in further detail below.

Biopsy devices according to the present invention are optionally configured as disposable devices.

Methods of obtaining a sample of biological material are provided according to embodiments of the present invention which include inserting into a biological material to be sampled a biopsy device having an elongated member having an external surface, a longitudinal axis, a distal end and a proximal end, and at least two protuberances extending from the elongated member, the protuberances oriented to provide low resistance entry of the distal end into the biological material and to provide higher resistance on withdrawal of the distal end from the biological material; and withdrawing the biopsy device.

Biological materials sampled using a biopsy device according to embodiment of the present invention include bodily tissues, tumors, cells, and other solid or semi-solid substances. Typically, a biopsy device described herein is used to obtain a sample of biological material from a subject, such as, but not limited to, a human. Biopsy devices described herein are used to obtain a sample of biological material from a non-human subject according to embodiments of the present invention, such as a companion animal including but not limited to dogs and cats; livestock including but not limited to cattle, horses, sheep, goats and poultry; and laboratory animals including but not limited to rodents.

Methods of obtaining a sample of biological material are provided according to embodiments of the present invention which include inserting into a biological material to be sampled a biopsy device having an elongated member having an external surface, a longitudinal axis, a distal end and a proximal end, and at least two protuberances extending from the elongated member, the protuberances oriented to provide low resistance entry of the distal end into the biological material and to provide higher resistance on rotation of the distal end in the biological material; rotating the biopsy device in the biological material and withdrawing the biopsy device.

Various actuation devices and supports can be used in conjunction with a biopsy device of the present invention. In particular embodiments, a biopsy needle is attached to an actuation device and/or support and inserted in the working channel of an endoscopic instrument.

According to embodiments of the present invention, an inventive component, such as a biopsy device described herein, has geometries previously unattainable and formed from a variety of compositions. An inventive component is characterized by a ratio of component length L to minimum component segment height Hₘᵢₙ to component segment minimal thickness Tₘᵢₙ of L:Hₘᵢₙ:Tₘᵢₙ of 20-80:1:0.5-5 for components in which Hₘᵢₙ is between 5 and 500 microns and the component is characterized by discontinuous edges. The component is formed from a variety of materials including metals, ceramics, alloys, and reinforced forms thereof to produce polycrystalline components of dimensions and compositions previously unattainable.

As used herein, “discontinuous edges” is defined as a sharp edge having a polygonal cross section in at least a portion of the component. The discontinuous edges as produced in an inventive component have an edge resolution of between 0.1 and 2 microns with feature resolutions as fine as 0.2 microns and commonly produced with a resolution of 2 microns for a particular feature.

As used herein, “edge resolution” is defined as the radius of curvature at an edge. Edge resolution is typically limited by grain size of a polycrystalline inventive component.

FIG. 1A depicts an inventive component generally at 10 where L is the total length of the component and L₁, L₂ and L₃ are the respective lengths of segments 1, 2 and 3. Each of the segments of component 10 has a respective height H₁, H₂ and H₃ as well as a respective thickness T₁, T₂ and T₃. In contrast to prior art lithographic formation techniques that are well suited for forming structures from silicon or silicon oxide with values of less than 20:1 that disfavor nonorthogonal angles α and β per FIG. 1A and edge resolutions that are poor, components according to the present invention are readily formed in reproducible batches from a variety of nanoparticulate precursors with improved attributes relative to lithographic formation techniques. An inventive component is also characterized by different dimensions in the component segments per FIG. 1A. This difference in segment dimensions is manifest in that component area in a plane of length L and height H, A_{LM} is optionally less than 40% of LH as a rectilinear area. A component area A_{LH} is readily formed with an area of 10 to 30% of LH with the lower end of this range corresponding to a component of higher curvature and comparative lesser height relative to the higher end of this range of component areas. FIG. 1B is a perspective view of an anvil component and a complementary cutter component according to the present invention. The cutter component has a lap joint between an arcuate cutting portion and a reinforcing portion sintered thereto. The anvil component and the cutting portion both have the dimensionality of an inventive component.

Manufacturing

The design and manufacturing process of an inventive component is illustrated in the flowchart shown in FIG. 2. Initially, material strength data is collected 22 and used as a constraint in the optimization routine, which calls on finite element analysis 24 to determine the optimal dimensions of the device. Once the optimal dimensions are found, the manufacturing process proceeds as follows: a) mold fabrication 26; b) colloid preparation 28; c) gel-casting slurry preparation 30; d) mold infiltration, gel-casting, and planarization 32; and e) mold removal and sintering 34. The final parts are filtered/cleaned 36 and optionally experimentally tested in order to update material strength properties. The procedure is then repeated based on design and material property improvements through subsequent iterations. By way of example, a zirconia test bar data initially indicated 671 MPa bend strength and is consistent with the requirements for the design of miniature forceps components as shown in FIG. 1A. Subsequent generations of material are showing strength improvement with zirconia test bars at ˜2400 MPa bend strength and stainless steel. Thus, additional feedback and subsequent process modification permits the design of improved surgical instruments and other components with the iterative design-manufacturing approach shown in FIG. 2.

Mold Fabrication

A polished polycrystalline alumina substrate 40 is used as substrate in this work in order to avoid the need to handle the small parts between processing steps. It is appreciated that operative substrates 40 are only limited by planarity and tolerance of the process conditions. Other operative substrates illustrative include silicon nitride, silicon carbide, glass, sapphire, YAG, nickel and alloy thereof, steel, titanium and alloys thereof, carbon, and soluble salt substrate. A substrate is chosen to preclude sintering of the inventive component thereto or any intervening layers to facilitate component lift of therefrom. A photoresist mold defining a mold cavity negative of a desired component or portion thereof is fabricated on top of the substrates using a modified UV lithography process. An exemplary photoresist operative herein for mold formation is SU8 (Microchem Corp.). It is appreciated that a positive or negative developing photoresist material is usable with a suitable mask.

Preferably, an antireflective coating 42 is spin-coated adjacent to the substrate 40 to eliminate scattered light from the reflective substrate surface to prevent curvature in the sidewalls of a mold cavity formed thereabove. An exemplary antireflective coating 42 operative herein is Barli-II 90 (Clariant Corp., Charlotte, N.C., USA). It is appreciated that any number of organic or inorganic antireflective materials are operative herein as antireflective coating 42. While organic antireflective materials tend to be applied with better coating uniformity through spin coating, inorganic material deposition often occurs with superior control over stoichiometry and thickness through gas phase deposition techniques of chemical vapor deposition (CVD), physical vapor deposition, atomic layer deposition, and enhanced versions thereof such as plasma-enhanced CVD, microwave-enhanced CVD, and the like. Exemplary of an inorganic antireflective material is dielectric silicon oxynitride. Optionally, an antireflective coating 42 is overcoating with an adhesion promoting layer 43 that promotes improved wetting by subsequent layer 44 relative to the antireflective coating 42.

An under layer of photoresist 44 is spin-coated to form the bottom of the mold. The under layer 44 is typically between 1 and 100 μm in thickness. The under layer 44 assures part separation from the substrate or the intermediate antireflective coating 42 before sintering and acts as a bottom surface of a mold cavity. A layer 46 of photoresist is applied using spin coating. The layer 46 forms sides of a component mold cavity while the under layer 44 forms a bottom surface thereof. Optionally, a second layer of photoresist 52 on glass is used as a UV light filter during exposure. The second layer of photoresist 52 minimizes the absorption of larger wavelengths by the mask layer 50 which creates undesirable trapezoidal cross-sections in the mold and cast parts. The photoresists used in the under layer 44, layer 46, and the second photoresist layer 52 are each independently the same photoresist or vary in properties such as viscosity, development type, development conditions, and dissolution conditions. Following exposure and post exposure baking, the mold layer is developed for instance in (propyleneglycol) monomethyletheracetate (PGMEA). FIG. 3 shows the lithography layering sequence. A hard bake such as the exemplary conditions of 180° C. for 20 minutes is used to fully crosslink the photoresist after development.

Colloid Preparation

Ceramic materials are attractive components on the micron scale components due to relatively high stress to failure and the ability to be easily formed into complex shapes via powder processing into polycrystalline components. Representative ceramics operative herein illustratively include silicon carbide, boron carbide, tungsten carbide, zirconia, titania, alumina, garnet structured oxides such as metal aluminum garnets, and spinel structured materials. Additionally, a ceramic is readily reinforced with particle and fiber fillers of metallic or inorganic materials. Representative fillers include high temperature metallic whiskers or particles, such as metals of stainless steel, tungsten and titanium, carbon nanotubes, and toughening ceramic materials such as Al₂O₃ and/or B₄C. An inventive component is also readily formed from metal powders to form an inventive component with a matrix formed of materials such as aluminum, steel, tungsten, titanium, nickel and alloys thereof. It is appreciated that an inventive component formed from a metal is also amenable to inclusion modification with particle or fiber fillers. Additionally, transformation toughened ceramic exhibits mechanical properties that are desirable in structural applications. In order to obtain micron scale resolution, the final grain size of the dense ceramic must be sub-micron, therefore dictating an initial particle size in the nanometer regime of between 1 and 1000 nanometers. Additionally, nanometer sized particles facilitate complex mold filling and edge resolution of between 0.1 and 2 microns. It is appreciated that nanophase particles exhibit a size dependent melting behavior that is approximately proportioned to the inverse of the particle radius, with the most pronounced size dependency observed in the 1 to 10 nanometer size regime. This attribute is readily exploited to produce an inventive component at a reduced sintering temperature, as compared to larger precursor particles.

Well dispersed, high solids loading slurries are required to fabricate dense parts using gel-casting. By way of example, yttria partially stabilized zirconia (Tosoh Corp. TZ-3Y) is dispersed and concentrated by chemically-aided attrition milling (CAAM). During CAAM, the as-received, spray dried commercial powder is added to DI water with ammonium polyacrylate at pH 8.5 (RT Vanderbuilt, Darvan 821A) on a 1.5 wt % dry basis as the dispersant, and milled using 1 mm zirconia media. Particle diameter based on the volume distribution is reduced from to 136 nm as measured by dynamic light scattering (Nano-S, Malvern Instruments, Southborough, Mass., USA), while electrosteric dispersion of the ceramic colloid is maintained at close particle separation distance by a high ζ-potential (−49 mV, ZetaPALS, Brookhaven Instruments Corp., Holtsville, N.Y., USA). This process of dispersion is readily practiced with any of the inventive component precursor materials with routine modifications to pH, weight percent dispersant, solvent, and polymer identity.

Gel-Casting Preparation

Gel-casting of the component particulate materials provides filling of the mold cavity with component precursor particulate retained in a cured polymer matrix that precludes the shrinkage induced cracking seen in mold filling with a particulate slurry. Additionally, a gel-casting has mechanical stability to withstand processing steps between mold cavity fill and particle sintering. Gel-casting involves mixture of component precursor particulate and optional fillers in a liquid organic monomer solvent that is amenable to mold cavity fill. The monomer mixture is polymerized in situ with the aid of a catalyst to form a gelled mold fill that is stronger than a slip cast green piece formed in the same mold cavity. Representative monomers are glycerol monoacrylates selected from the group of acrylic acid, hydroxymethylacrylamide, methacrylamide, methacrylic acid, methoxy (polyethylene glycol) monomethacrylate, n-vinyl pyrrolidone, acrylamide, alkyl-acrylamides, alkyl-acrylates, alkyl-methacrylamides, alkyl-methacrylates, dimethyl aminoethyl methacrylate, dimethyl aminopropyl methacrylamide, hydroxy-alkyl acrylamides, hydroxy-alkyl methacrylamides, hydroxy-alkyl acrylates, hydroxy-alkyl methacrylates, methacrylatoethyl trimethyl ammonium chloride, methacrylamidopropryl trimethyl ammonium chloride, p-styrene sulfonic acid, and p-styrene sulfonic acid salts. Catalysts for polymerization of monomers are conventional to the art and illustratively include persulfate-amine combinations.

A specific example of gel-casting in the present invention includes methacrylamide (Sigma-Aldrich) and N,N′-methylenebisacrylamide (Sigma-Aldrich) being used as the monomers for gel-casting in a 6:1 mass ratio. The total monomer content is 5 wt. % on a dry powder basis. The monomers are dissolved into the suspension of component particulate precursor using a vortex mixer (Scientific Industries, Vortex Genie 2). A 10:1 mass ratio of ammonium peroxydisulfate (Sigma-Aldrich) and N′,N′,N′,N′-tetramethylethylenediamine (Sigma-Aldrich) is used to initiate and catalyze the monomers, respectively. The initiator and catalyst are present at 2.5 wt % of the total monomer content.

Mold Infiltration, Gel-Casting, and Planarization

Prior to mold infiltration, the gelation reaction is initiated, leaving a working time. A working time of 5 min to 1 hour is typical but can readily be modified. The above gel-casting exemplary formulation provides a working time of approximately 25 min. The gelation reaction forms a network of cross-linked polymer between particles which provides additional green strength during the drying and mold removal steps. Following initiation, slurry is cast into the molds via a screen printing squeegee. A typical casting rate is 10 centimeters per second. Multiple passes with the squeegee are preferred to ensure complete mold filling with no entrapped air pockets, with the final squeegee pass preferably leaving a thin (1 mm) layer of excess slurry on top of the mold cavity. Gelation is preferably carried out under conditions to minimize drying and allow the reaction to be carried to completion. Representative conditions include a 100% relative humidity N₂ environment. Following gelation, the samples are allowed to dry, for example in ambient atmosphere. The excess slurry on top of the mold is removed until the mold surface becomes visible. An ethanol wipe is well suited to remove excess slurry. Dishing out of slurry from within the mold cavity is minimized to less than 5 μm. A cross-sectional view of a completed mold is illustrated in FIG. 4.

Mold Removal and Sintering

While mold removal is readily achieved through solvent removal with organic solvents, such developer or base such as KOH or pyrolysis, reactive ion etching (RIE) is preferably used to remove the entire mold without inducing the dimensional changes that are typically seen with pyrolysis or damage to the green ware filling the mold cavity. Representative RIE system parameters are set to 50 sccm O₂, 6 sccm SF_{S}, 350 W power, and an etch time of 75 minutes. Substrates are placed into a standard box furnace and sintered at 1° C./min to 300° C. with a 1 hr hold, 1° C./min to 450° C., 1° C./min to 600° C. with no hold, and 10° C./min to 1300° C. for 2 hrs with a furnace cool. Sintered components can be individually manipulated for further characterization or testing using a micromanipulator. FIG. 5 shows an exemplary fabricated zirconia test bar according to the present invention. It is appreciated that a sintered component exhibits a degree of shrinkage relative to the dimensions of the mold cavity. The shrinkage between a green ware mold fill and a sintered and densified material flows known ceramic densification principles and depends on factors including amount of gel polymer present, precursor particulate size, precursor particulate size distribution, and thermal time-temperature sintering profile.

This inventive manufacturing process is advantageous because components are fabricated with higher aspect ratios than previously available sharp edges (˜1 micron) while retaining a resolution of 2 microns in a mass production regime.

Still more complex three-dimensional articles are readily formed by lamination prior to RIE and densification. Lamination involves two dissimilar green components being brought into contact to allow sintering to form a composite component additive of the two mold cavity shapes in a given orientation along the overlapping interface between the components. In this way still more complex shapes are formed or an aspect ratio of L:Hₘᵢₙ:Tₘᵢₙ of 20-80:1:05-10 is achieved from separate elements that each do not satisfy this aspect ratio requirement. The sintering of two green ware mold fills after RIE is particularly well suited for the formation of lap joints between the green ware.

Micro Forceps Produced from Sintered Components

An inventive component as part of a pair of micro forceps is a monolithic compliant mechanism that uses large elastic deformation to achieve motion. Since compliant mechanisms can be monolithic they are ideal for small-scale applications because manufacturing and assembling of tiny parts are avoided. In addition, considering the nanoparticulate manufacturing process is currently limited to 2D parts, a compliant micro forceps is a good candidate for fabrication. The micro forceps consists of two parts, an upper and lower arm. FIG. 6 illustrates the basic geometry and actuation principle of the device based on the component shown in FIG. 1A.

The micro forceps is designed to fit inside the inner diameter (ID) of the outer sheath (shown in ghost) used for actuation. Here, tool dimensions should not require an outer sheath larger than 1 mm ID. As shown above, an outer sheath (made of medical grade stainless steel or other biocompatible material) encloses segment 1 of the device. As the sheath is advanced forward, the forceps arms are forced to displace toward one another, thus producing a grasping motion. The device is intended to grasp tissue, such as the gastric wall during NOTES procedures. The two component arms are separated and come into contact with one another as the sheath is advanced forward. This design feature results in stress relief as contact occurs between the forceps arms at point A.

Due to symmetry, one-half of the device is modeled as a cantilever beam undergoing large deformation. Geometric variables can be seen in FIG. 7, where L and L₁, L₂, and L₃ are defined with respect to FIG. 1A. Other variables are w=width (synonymous with T with respect to FIG. 1A), δₕₒₚ=half opening, δₕₛ=half separation distance and H₃=Height of segment 3. SEM images of such a component is shown in FIGS. 8A and 8B.

The distal tips of the micro forceps come into contact first when the device is closed, followed by the jaw surfaces (segment 3) gradually becoming parallel with additional pressure. Therefore, segment three is directed inward with dimensions L₃ and H₃ set to 0.075 mm and 0.05 mm, respectively. To simplify the number of geometric variables, L₁ is set to 30% of the total length (L) and δₕₛ is set to 0.015 mm. The thickness of the device into the page, T, is directly related to the thickness of the photoresist used as a mold. For this example, a 100 μm photoresist thickness is used for manufacturing. The final thickness is scaled by a thickness shrinkage factor due to the sintering process.

Any patents or publications mentioned in this specification are incorporated herein by reference to the same extent as if each individual publication is specifically and individually indicated to be incorporated by reference. U.S. patent application Ser. No. 12/534,329 filed Aug. 3, 2009; U.S. Provisional Patent Application Ser. No. 61/085,506 filed Aug. 1, 2008; U.S. Provisional Patent Application Ser. No. 61/237,959, filed Aug. 28, 2009; and U.S. Provisional Patent Application Ser. No. 61/150,568 filed Feb. 6, 2009 are all incorporated by reference herein in their entirety for all purposes.

The devices and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A biopsy device, comprising:
an elongated member having an external surface, a longitudinal axis, a distal end and a proximal end, and at least two protuberances extending from the elongated member, the protuberances oriented to provide low resistance entry of the distal end into a tissue or biological material and to provide higher resistance on withdrawal of the distal end from the tissue or biological material.

2. The biopsy device of claim 1, wherein the elongated member has a non-hollow cross-section along the length of the longitudinal axis.

3. The biopsy device of claim 1, wherein the elongated member comprises a working portion disposed towards the distal end of the elongated member and a support portion disposed proximally from the working portion, and wherein the protuberances are disposed on the working portion of the elongated member.

4. The biopsy device of claim 3, wherein the working portion of the elongated member is non-hollow.

5. The biopsy device of claim 1, wherein the elongated member is attached to an actuation device, the actuation device attached to a flexible insertion portion of an endoscopic instrument.

6. The biopsy device of claim 1, wherein the protuberances each have a front edge which tapers towards the distal end of the elongated member and a rear edge, a forward segment of the elongated member defined between the rear edge of each protuberance and the distal end and a rearward segment of the elongated member defined between the rear edge of each protuberance and the proximal end, wherein the rear edge is disposed so that at least a portion of the rear edge forms an angle which is 90° or less with the rearward segment, providing resistance when the biopsy device is removed from a bodily substance.

7. The biopsy device of claim 1, wherein the protuberances each have a top edge and a bottom edge, wherein the protuberances each have a longitudinal axis that is substantially parallel to the longitudinal axis of the elongated member, wherein the top and bottom edges of the protuberances extend from the elongated member, defining a space between the bottom edge of each protuberance and the external surface of the elongated member, providing resistance when the biopsy device is rotated in a bodily substance, thereby collecting biopsy material in the space and/or on the bottom edge.

8. The biopsy device of claim 3, wherein the working portion has a length in the range of about 1-14 millimeters, inclusive and a total diameter of the elongated member and protuberances in the range of about 0.5-6 millimeters, inclusive.

9. A biopsy device, comprising:
an elongated member having an external surface, a longitudinal axis, a distal end and a proximal end, and at least one helical protuberance extending radially from the elongated member, wherein the helical protuberance has a front edge which tapers towards the distal end of the elongated member and a rear edge, wherein the rear edge of the helical protuberance is disposed to form an angle which is 90° or less with the longitudinal axis of the elongated member, providing resistance when the biopsy device is removed from a bodily substance.

10. A method of obtaining a sample of biological material, comprising:
inserting into a biological material to be sampled a biopsy device having an elongated member having an external surface, a longitudinal axis, a distal end and a proximal end, and at least two protuberances extending from the elongated member, the protuberances oriented to provide low resistance entry of the distal end into the biological material and to provide higher resistance on withdrawal of the distal end from the biological material; and
withdrawing the biopsy device.

11. The method of claim 10, further comprising rotating the biopsy device in the biological material.

12. A process for forming a polycrystalline sintered mesoscale biopsy device, comprising:
filling a mold cavity formed in a photoresist with a mold fill, said mold fill comprising: a slurry of particles of ceramic, metal, or a combination thereof; and a polymerizable monomer in an amount to form a polymer that imparts strength to said mold fill, said mold cavity having a ratio of segment dimensions L:Hₘᵢₙ:Tₘᵢₙ of 20-80:1:0.5-10;
then removing said photoresist; and
heating said mold fill to remove said polymer.

13. The process of claim 12 wherein said slurry of particles have a particle diameter of from 5 nanometers to 500 microns and said mold cavity has a ratio of segment dimensions L:Hₘᵢₙ:Tₘᵢₙ of 20-80:1:0.5-10.

13. The process of claim 12 wherein said slurry of particles have a particle diameter of from 10 nanometers to 500 microns.

14. The process of claim 12 wherein said monomer is present from 0.5 to 20 weight percent of the weight of said particles.

15. The process of claim 12 wherein said removing said photoresist is by reactive ion etching.

16. The process of claim 12 further comprising removal of mold overburden prior to said removing said photoresist.

17. The process of claim 12 further comprising filling a second mold cavity formed in a second photoresist with a second slurry of particles of ceramic, metal, or a combination thereof and a second polymerizable monomer;
removing said second photoresist;
contacting said mold fill with said second mold fill to form an interface; and
heating said second mold fill in contact with said mold fill to sinter said mold fill with said second mold fill.

18. The process of claim 12 further comprising filling a third mold cavity formed in a third photoresist with a third slurry of particles of ceramic, metal, or a combination thereof and a third polymerizable monomer;
removing said third photoresist;
contacting said mold fill with said third mold fill to form a second interface; and
heating said third mold fill in contact with said second mold fill to sinter said second mold fill with said third mold fill.
